(19) United States
(12) Patent Application Publication
(10) Pub. No.: US 2023/0012135 A1
(43) Pub. Date: Jan. 12, 2023

(54) Composition for Promoting Relaxation and Methods of Making and Using the Same

(71) Applicant: Celesta Company LLC, Galesburg, MI (US)

(72) Inventors: David Wilfred Lafond, Galesburg, MI (US); Christine Lynn O'Neil, Jupiter, FL (US); Jean-Pierre Montmayeur, Atlanta, GA (US)

(21) Appl. No.: 17/855,324

(22) Filed: Jun. 30, 2022

Related U.S. Application Data
(60) Provisional application No. 63/218,453, filed on Jul. 5, 2021.

(51) Int. Cl. A61K 36/704; A61K 36/53; A23L 33/175

(52) U.S. Cl. CPC A61K 36/704; A61K 36/53; A23L 33/175

(57) ABSTRACT

Compositions are conducive to the promotion of a feeling of relaxation. The compositions of the present invention comprise a combination of hops extract, lemon balm extract, magnolia bark extract, and L-theanine to promote a feeling of relaxation in an individual. Methods of making and using the same are further provided.

### CROSS-REFERENCE TO RELATED APPLICATIONS

The present invention claims priority to U.S. Prov. Pat. App. No. 63/218,453 titled “Composition and Method for Promoting a Feeling of Relaxation,” filed Jul. 5, 2021, which is incorporated herein by reference in its entirety.

### TECHNICAL FIELD

The present invention relates to compositions conducive to the promotion of a feeling of relaxation. Specifically, the compositions of the present invention comprise a combination of hops extract, lemon balm extract, magnolia bark extract, and L-theanine to promote a feeling of relaxation in an individual. Methods of making and using the same are further provided.

### BACKGROUND

Many individuals have difficulty relaxing and dealing with stressors. Indeed, relaxation issues and stress management problems have increased over the past several years and will likely continue to increase with global lifestyle issues, such as extreme stress and increased screen time. Moreover, in modern society, the increasing use of stimulants and unhealthy diets combined with the prevalence of sensations of strain and emotional pressure can lead to feelings of fear and anxiety. Many individuals have lately experienced acute feelings of stress and anxiety due to the worldwide pandemic, economic downturn, global tension, and many other factors.

Psychological and physiological stress are known to impact immune and endocrine responses and pain modulation, as well as cognitive and cardiovascular function. Thus, the imposition of stress and anxiety on the human body can further lead to additional feelings of stress and anxiety in a feedback loop.

Pets also experience stress and high anxiety and can benefit from a regimen designed to promote and achieve states of relaxation. Specifically, many pets are left alone for extended periods of time. Pets may be especially stressed during holidays, when loud noises may be continuously heard throughout the celebrations, such as firecrackers, fireworks, guns, or other like noisemakers.

Drugs employed to reduce stress and manage anxiety, such as, for example, antidepressants, antihistamines, benzodiazepines, barbiturates, cannabinoids, opioids, and other like pharmaceuticals, can have unfavorable side effects such as intolerance, addiction, and withdrawal issues. Botanicals or nutraceutical alternatives to reduce stress and anxiety, thereby restoring balance and calm can help promote feelings of relaxation, without harmful side effects. However, botanicals and nutraceutical alternatives have heretofore not been sufficiently effective to achieve states of relaxation in individuals.

A need, therefore, exists for improved compositions and methods of making and using the same. Specifically, improved compositions and methods of making and using the same are needed to promote feelings of relaxation in a user. More specifically, improved compositions and methods of making and using the same are needed to allow a user to easily and effectively achieve a state of relaxation.

Moreover, a need exists for improved compositions and methods of making and using the same that is accomplished using various common ingredients. Specifically, a need exists for compositions and methods of making and using the same that utilizes a plurality of plant extracts or otherwise purified compounds extracted from plants. More specifically, a need exists for compositions and methods of using the same that utilizes a blend of at least four plant extracts or purified compounds combined together into a blend that may be easily administered or ingested by a user thereof.

In addition, a need exists for improved compositions and methods of making and using the same that is effective without harmful side effects. Specifically, a need exists for improved compositions and methods of making and using the same that will not lead to intolerance, dependency, or addiction issues, and/or withdrawal problems.

Further, a need exists for improved compositions and methods of making and using the same that may be utilized by humans as well as other creatures, such as pets. Specifically, a need exists for improved compositions and methods of making and using the same that may be in the form of beverages, foods, supplements, snacks, gummies, pet treats and pet food, tinctures, orally-dissolving film strips, or in other like forms that may be easily administered to a user thereof.

### SUMMARY OF THE INVENTION

The present invention relates to compositions conducive to the promotion of a feeling of relaxation. Specifically, the compositions of the present invention comprise a combination of hops extract, lemon balm extract, magnolia bark extract, and L-theanine to promote a feeling of relaxation in an individual. Methods of making and using the same are further provided.

To this end, in an embodiment of the present invention, a dietary composition formulated in a therapeutic effective amount to promote relaxation in humans or animals is provided. The dietary composition comprises a blend of: a first substance selected from the group of humulone, xanthohumole, myrcenol, and combinations thereof; rosmarinic acid; a second substance selected from the group of honokiol, magnolol, and combinations thereof; and L-theanine.

In an embodiment, the first substance is derived from hops.

In an embodiment, the first substance is from hops extract.

In an embodiment, the rosmarinic acid is from lemon balm extract.

In an embodiment, the second substance is derived from magnolia.

In an embodiment, the second substance is from magnolia bark extract.

In an embodiment, the first substance is from hops extract and the hops extract is present in the blend in an amount between about 0.1% and about 95%, wherein the rosmarinic acid is from lemon balm extract and the lemon balm extract is present in the blend in an amount between about 0.1% and 95%, wherein the second substance is from magnolia bark extract and the magnolia bark extract is present in the dietary blend in amount between about 0.1% and 90%, and L-theanine is present in the blend in an amount between about 0.1% and 95%.

In an embodiment, the hops extract is present in the blend in an amount between about 1% and about 20%, wherein the lemon balm extract is present in blend in an amount between about 10% and 75%, wherein the magnolia bark extract is present in the blend in an amount between about 1% and 25%, and wherein the L-theanine is present in the blend in an amount between about 1% and 40%.

In an embodiment, the second substance is from magnolia bark extract and further wherein the honokiol is present in the magnolia bark extract in an amount of about 2%.

In an embodiment, the first substance is from hops extract and the hops extract is present in the dietary composition in an amount between about 1 mg and about 1000 mg, wherein the rosmarinic acid is from lemon balm extract and the lemon balm extract is present in the dietary composition in an amount between about 10 mg and about 1000 mg, wherein the second substance is from magnolia bark extract and the magnolia bark extract is present in the dietary composition in amount between about 1 mg and about 1000 mg, and L-theanine is present in the dietary composition in an amount between about 1 mg and about 1000 mg.

In an embodiment, the hops extract is present in the dietary composition in an amount between about 10 mg and about 500 mg, wherein the lemon balm extract is present in the dietary composition in an amount between about 25 mg and 700 mg, wherein the magnolia bark extract is present in the dietary composition in an amount between about 5 mg and about 400 mg, and wherein the L-theanine is present in the dietary composition in amount between about 20 mg and about 600 mg.

In an embodiment, the hops extract is present in the dietary composition in an amount of about 20 mg, wherein the lemon balm extract is present in the dietary composition in an amount of about 400 mg, wherein the magnolia bark extract is present in the dietary composition in an amount of about 100 mg, and the L-theanine is present in the dietary composition in an amount of about 200 mg.

In an embodiment, the dietary composition is in the form of a pill, a capsule, a tablet, a softgel, a lozenge, a powder, a liquid, a gummy, or an orally-dissolving film strip.

In an embodiment, the dietary composition is in the form of a food or a beverage.

In an embodiment, the dietary composition provides an immediate therapeutic effect to promote relaxation.

In an embodiment, the dietary composition provides a sustained release therapeutic effect to promote relaxation.

In an alternate embodiment of the present invention, a method of promoting relaxation in humans or animals is provided. The method comprises the steps of: providing a dietary composition formulated in a therapeutic effective amount to promote relaxation in humans or animals comprising a first substance selected from the group of humulone, xanthohumole, myrcenol, and combinations thereof; rosmarinic acid; a second substance selected from the group of honokiol, magnolol, and combinations thereof; and L-theanine; and forming the dietary composition into an ingestible form.

In an embodiment, the first substance is from hops extract, the rosmarinic acid is from lemon balm extract, and the second substance is from magnolia bark extract.

In an embodiment, the hops extract is present in the dietary composition in an amount between about 10 mg and about 500 mg, the lemon balm extract is present in the dietary composition in an amount between about 25 mg and about 700 mg, the magnolia bark extract is present in the dietary composition in an amount between about 5 mg and about 400 mg, and the L-theanine is present in the dietary composition in an amount between about 20 mg and 600 mg.

In an embodiment, the form is a pill, a capsule, a tablet, a softgel, a lozenge, a powder, a liquid, a gummy, a beverage, a food, or an orally-dissolving film strip.

It is, therefore, an advantage and objective of the present invention to provide improved compositions and methods of making and using the same.

Specifically, it is an advantage and objective of the present invention to provide improved compositions and methods of making and using the same to promote feelings of relaxation in a user.

More specifically, it is an advantage and objective of the present invention to provide improved compositions and methods of making and using the same to allow a user to easily and effectively achieve a state of relaxation.

Moreover, it is an advantage and objective of the present invention to provide improved compositions and methods of making and using the same that is accomplished using various common ingredients.

Specifically, it is an advantage and objective of the present invention to provide compositions and methods of making and using the same that utilizes a plurality of plant extracts or otherwise purified compounds extracted from plants.

More specifically, it is an advantage and objective of the present invention to provide compositions and methods of using the same that utilizes a blend of at least four plant extracts or purified compounds combined together into a blend that may be easily administered or ingested by a user thereof.

In addition, it is an advantage and objective of the present invention to provide improved compositions and methods of making and using the same that is effective without harmful side effects.

Specifically, it is an advantage and objective of the present invention to provide improved compositions and methods of making and using the same that will not lead to intolerance, dependency or addiction issues, and/or withdrawal problems.

Further, it is an advantage and objective of the present invention to provide improved compositions and methods of making and using the same that may be utilized by humans as well as other creatures, such as pets.

Specifically, it is an advantage and objective of the present invention to provide improved compositions and methods of making and using the same that may be in the form of beverages, foods, supplements, snacks, pet treats and pet food, tinctures, pills, gummies, tablets, orally-dissolving film strips, or in other like forms that may be easily administered to a user thereof.

Additional features and advantages of the present invention are described in, and will be apparent from, the detailed description of the presently preferred embodiments and from the drawings.

### DETAILED DESCRIPTION OF THE PRESENTLY PREFERRED EMBODIMENTS

The present invention relates to compositions conducive to the promotion of a feeling of relaxation. Specifically, the compositions of the present invention comprise a combination of hops extract preferably comprising humulone, xanthohumole and/or myrcenol, lemon balm extract comprising an amount of rosmarinic acid, magnolia bark extract preferably comprising an amount of honokiol and/or an amount of magnolol, and L-theanine to promote a feeling of relaxation in an individual. Methods of making and using the same are further provided.

For the purposes of the present invention, the term “relaxation” is defined as the feeling of reduced anxiety and increased well-being.

The composition of the present invention, described herein, is designed to target biological components promoting and supporting a feeling of relaxation, including the hypothalamus-pituitary axis and Gamma-aminobutyric acid (GABA) systems while supporting mood stabilization.

The composition of the present invention comprises various amounts of four plant extracts or ingredients (i.e., purified compounds derived from plants) that are combined together into a blend that is designed to promote a feeling of relaxation.

Preferably, the present invention comprises a blend of ingredients that is soluble in aqueous media. Specifically, the composition of the present invention is preferably administered in the form of a beverage or a food that may be ingestible by a user thereof. Particularly preferred exemplary foods may include frozen novelties and snack bars for humans, or pet foods and pet treats for animals. Other forms of the composition of the present invention may include pills, capsules, tablets, softgels, lozenges, powders, liquids, gummies, or orally-dissolving film strips. The present invention as described herein may be ingested by humans and/or animals, such as pets.

In an embodiment of the present invention, the composition may comprise a blend of a substance selected from the group of humulone, xanthohumole, myrcenol, and combinations thereof, rosmarinic acid, honokiol and/or magnolol, and L-theanine in therapeutic effective amounts to promote a feeling of relaxation in humans and/or animals after ingesting the same. Specifically, the substance may be derived from hops, and preferably is present in hops extract that may be incorporated into the composition. In a preferred embodiment, the substance comprises a combination of humulone, xanthohumole, and myrcenol. Moreover, the rosmarinic acid may preferably be present in lemon balm extract that may be incorporated into the composition. In addition, the honokiol and/or magnolol may be derived from magnolia, and preferably is present in magnolia bark extract that may be incorporated into the composition.

In a preferred embodiment of the present invention, the composition may comprise a blend of hops extract, lemon balm extract, magnolia extract, and L-Theanine in therapeutically effective amounts to promote a feeling of relaxation after ingesting the same. Specifically, the composition may comprise a blend thereof of the four ingredients listed above (hereinafter known as a blend of the four active ingredients) in the following ranges: between about 0.1% and about 95% hops extract, between about 0.1% and about 95% lemon balm extract, between about 0.1% and about 90% magnolia extract, and between about 0.1% and about 95% L-Theanine. More preferably, the composition may comprise a blend thereof of the four ingredients listed above in the following ranges: between about 1% to about 20% hops extract, between about 10% and about 75% lemon balm extract, between about 1% and about 25% magnolia extract, and between about 1% and about 40% L-Theanine. Most preferably, the composition may comprise a blend thereof of the four ingredients listed above in the following amounts: about 3% hops extract, about 55% lemon balm extract, about 14% magnolia extract, and about 28% L-Theanine.

The compositions of the present invention may further comprise excipients and/or emulsifiers, sweeteners, bulking agents, mouthfeel and/or flavor agents, and other agents or ingredients suitable for beverage food and/or beverage compositions. It should be noted that the amounts and percentages preferred in the present invention are measured against the total of the blend of the active ingredients listed above without regard to additional ingredients are provided in the compositions described herein.

### Hops

Hops (Humulus lupulus) is an important agricultural perennial plant that has been traditionally used to flavor beer. Hops flavors for beer are typically produced from the cones and flowers of the plant, which are rich in flavonoids, phenolic acids, aromatic oils, tannins, and resins. Besides its use in the beer industry, hops extracts have been used as sedatives to reduce anxiety and help sleep. Pre-clinical trial studies both in animals and cellular models have demonstrated sleep enhancing and anxiolytic activity presumably by interacting with the GABA-A and serotonin systems, which are known to be involved in the sleep process. In humans, hops in polyherbal blends of plants known for their sedative properties has been shown to be effective to improve sleep quality. A combination of Valerian and hops has been shown to be particularly effective by acting concomitantly on the adenosine, melatonin, and GABA-A systems that regulate sleep.

In an embodiment of the present invention, the composition may include hops extract, specifically hops cone extract. A serving of the composition may include from about 1 mg to about 1000 mg of hops cone extract. More preferably, a serving of the composition may include from about 10 mg to about 500 mg of hops cone extract. In a preferred embodiment, the composition may comprise about 20 mg hops cone extract per serving.

The hops extract of the present invention comprises at least one substance selected from the group of humulone, xanthohumole, and myrcenol. Preferably, the hops extract comprises a combination of humulone, xanthohumole, and myrcenol. It should be noted that a dietary composition of the present may be formulated using purified humulone, xanthohumole, and/or myrcenol not in the form of hops extract or hops cone extract.

### Lemon Balm Extract

Lemon balm (Melissa officinalis) is a perennial plant of the mint family native of Europe and Asia. Lemon balm has been used in traditional medicine to reduce stress and anxiety and treat intestinal discomfort. It is known for its calming properties and is thought to produce relaxation effects through central modulation of both nicotinic and muscarinic acetylcholine receptor activity.

Calming effects were observed in randomized, placebo-controlled, double-blind clinical studies investigating the acute effects of Lemon balm extract on cognition and mood. Another clinical study also demonstrated properties to reduce the mood effects of laboratory-induced stress in humans.

In an embodiment of the present invention, the composition may comprise from about 10 mg to about 1,000 mg of lemon balm extract per serving to promote a feeling of relaxation. More preferably, the composition may comprise from about 25 mg to about 700 mg lemon balm extract. The preferred dosage of lemon balm extract in the present invention comprises about 400 mg.

The lemon balm extract of the present invention comprises rosmarinic acid, which is believed to be an active ingredient of the composition of the present invention. It should be noted that a dietary composition of the present may be formulated using purified rosmarinic acid not in the form of lemon balm extract.

### Magnolia Extract

Magnolia (Magnolia officinalis) is a tree native to China. Its bark extract has been used in traditional Chinese Medicine for its sedative and anxiolytic actions. Two major bioactive constituents of Magnolia bark extract which have been characterized are magnolol and honokiol. Both magnolol and honokiol, through their modulation of the benzodiazepine site of the GABA-A receptor, have been demonstrated, in mice, to shorten the sleep latency to non-rapid eye movement (non-REM, NREM) sleep while increasing the amount of NREM sleep. REM sleep amount remained about the same. The relaxation and sedative effects of Magnolia extract have been further demonstrated in humans. In addition, both compounds also modulate the cannabinoid and adenosine systems.

In an embodiment, a composition herein includes magnolia bark extract to improve relaxation. A serving of the said composition includes from about 1 mg to about 1000 mg of magnolia. More preferably, a serving of the composition may include from about 5 mg to about 400 mg magnolia. The preferred dosage of said composition comprises about 100 mg of magnolia.

The magnolia extract of the present invention comprises honokiol, preferably in an amount of about 2%, and magnolol, each of which is believed to be an active ingredient of the composition of the present invention. It should be noted that a dietary composition of the present may be formulated using purified honokiol and/or magnolol not in the form of magnolia extract.

### L-Theanine

L-theanine (N-ethyl-L-glutamine), also known as “theanine,” is an amino acid abundant in tea leaves and also present in some mushrooms. Green tea (Camellia sinensis) is well known in Traditional Chinese Medicine for its sedative effect. In animal models theanine reduces markers of stress such as blood pressure. In humans, administration of theanine resulted in a reduction in heart rate and salivary immunoglobulin A (s-IgA) during an acute stress task. This ability to induce relaxation and reduce psychological stress confirmed by the production of alpha brain waves soon after ingestion, is conducive to sedation and sleep as several studies demonstrate. This effect seems to be related to the modulation of a wide range of neurotransmitters, and in particular, glutamate.

In an embodiment of the present invention the composition may comprise from about 10 mg to about 1000 mg of L-theanine. More preferably, the composition of the present invention may comprise from about 20 mg to about 600 mg L-theanine. The preferred dosage of L-theanine in the present composition comprises about 200 mg per serving.

### EXAMPLES

### Dietary Composition Example

In an embodiment of the present invention, a dietary Composition comprises a blend of active ingredients, as described herein in the following amounts, as shown by Table 1:

TABLE 1

| Ingredient | Active ingredients | Amount |
|---|---|---|
| Magnolia bark extract | 2% honokiol, magnolol | 100 mg |
| Hops extract | Humulone, xanthohumole, | 20 mg |
| | and myrcenol | |
| L-theanine | | 200 mg |
| Lemon balm extract | Rosmarinic acid | 400 mg |

### Beverage Example

In an embodiment of the present invention, a relaxation beverage example was formulated in the following amounts, as shown by Table 2:

TABLE 2

| | | | Percent of |
|---|---|---|---|
| | | Percent | total blend |
| | Active | of total | of the active |
| Ingredient | ingredients | beverage | ingredients |
| Juice concentrate | | 2.71 | n/a |
| Citric acid | | 0.12 | n/a |
| Sugar | | 3.1 | n/a |
| Stabilizer | | 0.3 | n/a |
| Stevia | | 3.2 | n/a |
| Flavor | | 0.59 | n/a |
| Magnolia bark | 2% honokiol, | 0.04 | 14 |
| extract | magnolol | | |
| Hops extract | Humulone, | 0.01 | 3 |
| | xanthohumole, | | |
| | and myrcenol | | |
| L-theanine | | 0.09 | 28 |
| Lemon balm extract | Rosmarinic acid | 0.18 | 55 |
| Water | | 89.66 | n/a |

The above-identified ingredients were blended together to form a Sangria-flavored beverage.

### Water Modifying Powder Example

In an embodiment of the present invention, a water modifying powder example was formulated in the following amounts, as shown by Table 3:

TABLE 3

| | | | Percent of |
|---|---|---|---|
| | | Percent | total blend |
| | Active | of total | of the active |
| Ingredient | ingredients | powder | ingredients |
| Flavor | | 19.92 | n/a |
| Citric acid | | 8.83 | n/a |
| Monk fruit 50% | | 0.12 | n/a |
| Stevia Reb A | | 0.12 | n/a |
| Erythritol | | 61.68 | n/a |
| Magnolia bark | 2% honokiol, | 1.30 | 14 |
| extract | magnolol | | |
| Hops extract | Humulone, | 0.26 | 3 |
| | xanthohumole, | | |
| | and myrcenol | | |
| L-theanine | | 2.60 | 28 |
| Lemon balm extract | Rosmarinic acid | 5.19 | 55 |

The above-identified ingredients were blended together to form a powder for dispensing in an amount of water for drinking.

### Gummy Example

In an embodiment of the present invention, a gummy was formulated in the following amounts, as shown by Table 4:

TABLE 4

| | | | Percent of |
|---|---|---|---|
| | | Percent | total blend |
| | Active | of total | of the active |
| Ingredient | ingredients | Gummy | ingredients |
| Water | | 33.25 | n/a |
| Pectin type D slow set | | 0.72 | n/a |
| Sucrose | | 28.39 | n/a |
| Glucose syrup 42DE | | 15.74 | n/a |
| Isofructose Syrup | | 10.10 | n/a |
| Sodium citrate 2H20 | | 0.07 | n/a |
| Gelatin 150 bloom | | 7.80 | n/a |
| Citric Acid (50% sol w/v) | | 3.00 | n/a |
| Magnolia bark extract | 2% honokiol, | 1.33 | 14 |
| | magnolol | | |
| Hops extract | Humulone, | 0.27 | 3 |
| | xanthohumole, | | |
| | and myrcenol | | |
| L-theanine | | 2.67 | 28 |
| Lemon balm extract | Rosmarinic acid | 5.33 | 55 |
| (Evaporation) | | −8.66 | n/a |

The above-identified ingredients were blended together to form a gummy for ingestion. A serving size included two gummies of approximately 9 grams each.

It should be noted that various changes and modifications to the presently preferred embodiments described herein will be apparent to those skilled in the art. Such changes and modifications may be made without departing from the spirit and scope of the present invention and without diminishing its attendant advantages. Further, references throughout the specification to “the invention” are nonlimiting, and it should be noted that claim limitations presented herein are not meant to describe the invention as a whole. Moreover, the invention illustratively disclosed herein suitably may be practiced in the absence of any element which is not specifically disclosed herein.

I claim:

1. A dietary composition formulated in a therapeutic effective amount to promote relaxation in humans or animals comprising a blend of:
a first substance selected from the group of humulone, xanthohumole, myrcenol, and combinations thereof;
rosmarinic acid;
a second substance selected from the group of honokiol, magnolol, and combinations thereof; and
L-theanine.

2. The dietary composition of claim 1 wherein the first substance is derived from hops.

3. The dietary composition of claim 1 wherein the first substance is from hops extract.

4. The dietary composition of claim 1 wherein the rosmarinic acid is from lemon balm extract.

5. The dietary composition of claim 1 wherein the second substance is derived from magnolia.

6. The dietary composition of claim 1 wherein the second substance is from magnolia bark extract.

7. The dietary composition of claim 1 wherein the first substance is from hops extract and the hops extract is present in the blend in an amount between about 0.1% and about 95%, wherein the rosmarinic acid is from lemon balm extract and the lemon balm extract is present in the blend in an amount between about 0.1% and 95%, wherein the second substance is from magnolia bark extract and the magnolia bark extract is present in the dietary blend in amount between about 0.1% and 90%, and L-theanine is present in the blend in an amount between about 0.1% and 95%.

8. The dietary composition of claim 7 wherein the hops extract is present in the blend in an amount between about 1% and about 20%, wherein the lemon balm extract is present in blend in an amount between about 10% and 75%, wherein the magnolia bark extract is present in the blend in an amount between about 1% and 25%, and wherein the L-theanine is present in the blend in an amount between about 1% and 40%.

9. The dietary composition of claim 1 wherein the second substance is from magnolia bark extract and further wherein the honokiol is present in the magnolia bark extract in an amount of about 2%.

10. The dietary composition of claim 1 wherein the first substance is from hops extract and the hops extract is present in the dietary composition in an amount between about 1 mg and about 1000 mg, wherein the rosmarinic acid is from lemon balm extract and the lemon balm extract is present in the dietary composition in an amount between about 10 mg and about 1000 mg, wherein the second substance is from magnolia bark extract and the magnolia bark extract is present in the dietary composition in amount between about 1 mg and about 1000 mg, and L-theanine is present in the dietary composition in an amount between about 1 mg and about 1000 mg.

11. The dietary composition of claim 10 wherein the hops extract is present in the dietary composition in an amount between about 10 mg and about 500 mg, wherein the lemon balm extract is present in the dietary composition in an amount between about 25 mg and 700 mg, wherein the magnolia bark extract is present in the dietary composition in an amount between about 5 mg and about 400 mg, and wherein the L-theanine is present in the dietary composition in amount between about 20 mg and about 600 mg.

12. The dietary composition of claim 10 wherein the hops extract is present in the dietary composition in an amount of about 20 mg, wherein the lemon balm extract is present in the dietary composition in an amount of about 400 mg, wherein the magnolia bark extract is present in the dietary composition in an amount of about 100 mg, and the L-theanine is present in the dietary composition in an amount of about 200 mg.

13. The dietary composition of claim 1 wherein the dietary composition is in the form of a pill, a capsule, a tablet, a softgel, a lozenge, a powder, a liquid, a gummy, or an orally-dissolving film strip.

14. The dietary composition of claim 1 wherein the dietary composition is in the form of a food or a beverage.

15. The dietary composition of claim 1 wherein the dietary composition provides an immediate therapeutic effect to promote relaxation.

16. The dietary composition of claim 1 wherein the dietary composition provides a sustained release therapeutic effect to promote relaxation.

17. A method of promoting relaxation in humans or animals comprising the steps of:
providing a dietary composition formulated in a therapeutic effective amount to promote relaxation in humans or animals comprising a first substance selected from the group of humulone, xanthohumole, myrcenol, and combinations thereof; rosmarinic acid; a second substance selected from the group of honokiol, magnolol, and combinations thereof; and L-theanine; and
forming the dietary composition into an ingestible form.

18. The method of claim 17 wherein the first substance is from hops extract, the rosmarinic acid is from lemon balm extract, and the second substance is from magnolia bark extract.

19. The method of claim 18 wherein the hops extract is present in the dietary composition in an amount between about 10 mg and about 500 mg, the lemon balm extract is present in the dietary composition in an amount between about 25 mg and about 700 mg, the magnolia bark extract is present in the dietary composition in an amount between about 5 mg and about 400 mg, and the L-theanine is present in the dietary composition in an amount between about 20 mg and 600 mg.

20. The method of claim 17 wherein the form is a pill, a capsule, a tablet, a softgel, a lozenge, a powder, a liquid, a gummy, a beverage, a food, or an orally-dissolving film strip.
